(19) United States
(12) Patent Application Publication
(10) Pub. No.: US 2011/0136245 A1
(43) Pub. Date: Jun. 9, 2011

(54) SYNERGISTIC INTERACTIONS OF PHENOLIC COMPOUNDS FOUND IN FOOD

(76) Inventor: Tory L. Parker, Spanish Fork, UT (US)

(21) Appl. No.: 12/907,681

(22) Filed: Oct. 19, 2010

Related U.S. Application Data
(60) Provisional application No. 61/279,368, filed on Oct. 20, 2009.
(60) Provisional application No. 61/339,244, filed on Mar. 2, 2010.
(60) Provisional application No. 61/399,548, filed on Jul. 14, 2010.

(51) Int. Cl. G01N 33/50

(52) U.S. Cl. 436/94; 436/129; 436/93

(57) ABSTRACT

Synergistic nutritional supplements of multiple antioxidant compounds with ratios derived from the ratios in naturally occurring foodstuffs.

### CROSS REFERENCE TO RELATED APPLICATIONS

Benefit is claimed from U.S. Provisional Patent Application 61/279,368, filed Oct. 20, 2009; U.S. Provisional Patent Application 61/339,244, filed Mar. 2, 2010; and U.S. Provisional Patent Application 61/399,548, filed Jul. 14, 2010, which are hereby incorporated by reference

### BACKGROUND

Plants produce phenolic compounds to act as cell signaling molecules, antioxidants, or toxins to invading pests (Crozier and others 2006). Research has explored the components of fruit (Robards and others 1999; Franke and others 2004; Harnly and others 2006), with primary emphasis being placed on phenolic compounds because of their high antioxidant capacity.

There is a discrepancy between the antioxidant capacity of an individual phenolic compound at the concentration found in fruit and the antioxidant capacity of the whole fruit (Miller and Rice-Evans 1997; Zheng and Wang 2003); the antioxidant capacity of the whole fruit is higher. Possible explanations for the difference could include unidentified compounds in the fruit, the sum total of many compounds present in the fruit at low concentration, or synergistic interactions between phenolic compounds.

Lila and Raskin (2005) discussed additive or synergistic potentiation in terms of endointeractions, or interactions within a plant that may modify its pharmacological effects, and exointeractions, which are interactions between unrelated plant components and/or drugs. Antioxidant synergism through exointeractions has received some attention. Yang and Liu (2009) reported that the combination of an apple extract and quercetin 3-β-D-Glucoside exhibits synergistic antiproliferative activity toward human breast cancer cells. The combination of soy and alfalfa phytoestrogen extracts and acerola cherry extracts works synergistically to inhibit LDL oxidation in vitro (Hwang and others 2001). Liao and Yin (2000) demonstrated that combinations of alpha-tocopherol and/or ascorbic acid with caffeic acid, catechin, epicatechin, myricetin, gallic acid, quercetin, and rutin had greater antioxidant activity than any of the compounds alone in an Fe2+-induced lipid oxidation system.

There is a current interest in developing or discovering effective natural preservatives (Galal 2006). Approaches include the use of extracts (Serra and others 2008; Conte and others 2009), phenolic compounds (Rodr'iguez Vaquero and Nadra 2008), or mixtures of compounds (Oliveira and others 2010) as antimicrobial agents. Understanding the mechanisms behind the functionality of potential antioxidant mixtures is important to their potential development as preservatives.

### REFERENCES

Amie, D, Davidovi, cD, Be.sloD, RastijaV, Lu.c B, Trinajsti, of the antioxidant activity of flavonoids. Curr Med Chem 14:827-45.
Bravo, L., 1998. Polyphenols: Chemistry, dietary sources, metabolism, and nutritional significance. Nutrition Reviews 56, 317-333.
Buettner G R. 1993. The pecking order of free radicals and antioxidants: lipid peroxidation, alpha-tocopherol, and ascorbate. Arch Biochem Biophys 300:535-43.
Conte A, Scrocco C, Sinigaglia M, Del Nobile M A. 2009. Lemon extract as natural preservative in fruit salad. J Food Saf 29:601-16.
Crozier A, Clifford M N, Ashihara H, eds. 2006. Plant secondary metabolites. Oxford: Blackwell Publishing. 372 p.
Cuvelier C, Bondet V, Berset C. 2000. Behavior of phenolic antioxidants in a partitioned medium: structure-activity relationship. J Am Oil Chem Soc 77:819-23.
Davalos A, Gomez-Cordoves C, Bartolome B. 2004. Extending Applicability of the Oxygen Radical Absorbance Capacity (ORAC-Fluorescein) Assay. J Agric Food Chem 52:48-54.
Delgado-Vargas, F.; Jiménez, A. R.; Paredes-López, O., 2000. Natural pigments: Carotenoids, anthocyanins, and betalains—Characteristics, biosynthesis, processing, and stability. Critical Reviews in Food Science and Nutrition 40, 173-289.
Di Majo D, Giammanco M, La Guardia M, Tripoli E, Giammanco S, Finotti E. 2005. Flavanones in citrus fruit: structure-antioxidant activity relationships. Food Res Int 38:1161-6.
Foley S, Navaratnam S, McGarvey D J, Land E J, Truscott T G, Rice-Evans C A. 1999. Singlet oxygen quenching and the redox properties of hydroxycinnamic acids. Free Radic Biol Med 26:1202-208.
Franke A, Custer L, Arakaki C, Murphy S. 2004. Vitamin C and flavonoid levels of fruits and vegetables consumed in Hawaii. J Food Compost Anal 17:1-35.
Frankel E N, Huang S W, Kanner J, German J B. 1994. Interfacial phenomena in the evaluation of antioxidants: bulk oil vs. emulsions. J Agric Food Chem 42:1054-9.
Freeman, B.; Eggett, D.; Parker, T. Synergistic and antagonistic interactions of phenolic compounds found in navel oranges. J. Food. Sci. 2010, in press.
Galal A M. 2006. Natural product-based phenolic and nonphenolic antimicrobial food preservatives and 1,2,3,4-tetrahydroxybenzene as a highly effective representative: a review of patent literature 2000-2005. Recent Pat Antiinfect Drug Discov 1:231-9.
Gitz, D.; Liu, L.; McClure, J. Phenolic metabolism, growth, and uv-b tolerance in phenylalanine ammonia-lyase-inhibited red cabbage. Phytochemistry. 1998, 49, 377-386
Halliwell, B., Zhao, K., Whiteman, M., 2000. The gastrointestinal tract: A major site of antioxidant action? Free Radicals Research 33, 819-830.
Harnly J M, Doherty R F, Beecher G R, Holden J M, Haytowitz D B, Bhagwat S, Gebhardt S. 2006. Flavonoid content of U.S. fruits, vegetables and nuts. J Agric Food Chem 54:9966-77.
Hidalgo, M.; Sánchez-Moreno, C.; de Pascual-Teresa, S., 2010. Flavonoid-flavonoid interaction and its effect on their antioxidant activity. Food Chemistry 121, 691-696.
Hwang J, Hodis H N, Sevanian A. 2001. Soy and alfalfa phytoestrogen extracts become potent low-density lipoprotein antioxidants in the presence of acerola cherry extract. J Agric Food Chem 49:308-14.
Jørgensen L V, Skibsted L H. 1998. Flavonoid deactivation of ferrylmyoglobin in relation to ease of oxidation as determined by cyclic voltammetry. Free Radic Res 28:335-51.
Jovanovic S V, Steenken S, Tosic M, Marjanovic B, Simic M G. 1994. Flavonoids as antioxidants. J Am Chem Soc 116:4846-51.
Koga T, Terao J. 1995. Phospholipids increase radical-scavenging activity of vitamin E in a bulk oil model system. J Agric Food Chem 43:1450-4.
Liao K, Yin M. 2000. Individual and combined antioxidant effects of seven phenolic agents in human erythrocyte membrane ghosts and phosphatidylcholine liposome systems: importance of the partition coefficient. J Agric Food Chem 48:2266-70.
Lila M A, Raskin I. 2005. Health-related interactions of phytochemicals. J Food Sci 70:R20-7.
Miller N J, Rice-Evans C A. 1997. The relative contributions of ascorbic acid and phenolic antioxidants to the total antioxidant activity of orange and apple fruit juices and blackcurrant drink. Food Chem 60:331-7.
Oliveira C E V, de Stamford T L M, Gomes Neto N J, de Souza E L. 2010. Inhibition of Staphylococcus aureus in broth and meat broth using synergies of phenolics and organic acids. Int J Food Microbiol 137:312-16.
Osmani, S. A.; Hansen, E. H.; Malien-Aubert, C.; Olsen, C.-E.; Bak, S.; Møller, B. L., 2009. Effect of glucuronosylation on anthocyanin color stability. Journal of Agricultural and Food Chemistry 57, 3149-3155.
Ou B, Huang D, Hampsch-Woodill M, Flanagan J A, Deemer E K. 2002. Analysis of antioxidant activities of common vegetables employing oxygen radical absorbance capacity (ORAC) and ferric reducing antioxidant power (FRAP) assays: a comparative study. J Agric Food Chem 50:3122-8.
Parker T L, Miller S A, Myers L E, Miguez F E, Engeseth N J. 2010. Evaluation of synergistic antioxidant potential of complex mixtures using oxygen radical absorbance capacity (ORAC) and electron paramagnetic resonance (EPR). J Agric Food Chem 58:209-17.
Peyrat-Maillard M N, Cuvelier M E, Berset C. 2003. Antioxidant activity of phenolic compounds in 2,2′-azobis(2-amidinopropane) dihydrochloride (AAPH)-induced oxidation: synergistic and antagonistic effects. J Am Oil Chem Soc 80:1007-12.
Proteggente A, Saija A, De Pasquale A, Rice-Evans C. 2003. The compositional characterisation and antioxidant activity of fresh juices from sicilian sweet (Citrus sinensis L. Osbeck) varieties. Free Radic Res 37:681-7.
Rice-Evans C A. 2001. Flavonoid antioxidants. Curr Med Chem 8:797-807.
Rice-Evans, C., Miller, N., Paganga, G., 1996. Structure-antioxidant activity relationships between flavonoids and phenolic acids. Free Radicals in Biology and Medicine 20, 933-956.
Robards K, Prenzler P D, Tucker G, Swatsitang P, Glover W. 1999. Phenolic compounds and their role in oxidative processes in fruits. Food Chem 66:401-36.
Rodr'iguez Vaquero M J, Nadra M C M de. 2008. Growth parameter and viability modifications of Escherichia coli by phenolic compounds and Argentine wine extracts. Appl Biochem Biotechnol 151:342-52.
Serra A T, Matias A A, Nunes A V M, Leit~ao M C, Brito D, Bronze R, Silva S, Pires A, Crespo M T, San Romao M V, Duarte C M. 2008. In vitro evaluation of olive- and grape-based natural extracts as potential preservatives for food. Innov Food Sci Emerg Technol 9:311-19.
U.S. Department of Agriculture, A Database for the Flavonoid Content of Selected Foods [Internet]. Release 2.1. Beltsville, Md.: U.S. Department of Agriculture; c2007. Available from: http://www.ars.usda.gov/nutrientdata. Accessed Mar. 15, 2010.
U.S. Department of Agriculture, A Database for the Flavonoid Content of Selected Foods. http://www.nal.usda.gov/fnic/foodcomp
U.S. Department of Agriculture, Agricultural Research Service. (2007) USDA Database for the Flavonoid Content of Selected Foods, Release 2.1. Retrieved March 2010 from: http://www.ars.usda.gov/Services/docs.htm?docid=6231
U.S. Department of Agriculture, Agricultural Research Service. (2010). Oxygen Radical Absorbance Capacity of Selected Foods, Release 2. Retrieved March 2010 from: http://www.ars.usda.gov/Services/docs.htm?docid=15866
van't Veer, P.; Jansen, M. C. J. F.; Klerk, M.; Kok, F. J. Fruits and vegetables in the prevention of cancer and cardiovascular disease. Public Health Nutr. 2000, 3, 103-107.
Yang J, Liu R H. 2009. Synergistic effect of apple extracts and quercetin 3-β-D-glucoside combination on antiproliferative activity in MCF-7 human breast cancer cells in vitro. J Agric Food Chem 57:8581-6.
Zhao, Y., 2007. Berry Fruit: Value-Added Products for Health Promotion. (pp. 152-153). Boca Raton, Fla., USA: CRC Press.
Zheng W, Wang S Y. 2003. Oxygen radical absorbing capacity of phenolics in blueberries, cranberries, chokeberries, and lingonberries. J Agric Food Chem 51:502-9.

### SUMMARY

Phenolic compounds are known to have antioxidant and antimicrobial properties. These properties may be useful in the preservation of foods or beverages. The interactive antioxidant capacity of phenolic compounds within foods has not been well explored. Understanding how combinations of fruit antioxidants work together will support their future use in preservation of foods and/or beverages.

An aspect is the discovery that synergistic combinations of antioxidant phenolic compounds exist in foodstuffs. The discovery that synergistic endointeractions occur between the antioxidants themselves is significant. Another aspect is a system for determining synergistic combinations of antioxidants, and the discovery that the synergism depends in part of the ratios at which these antioxidant compounds are present in the mixture.

Another aspect is using food-stuffs, such as fruit, as model for determining possible synergistic antioxidant combinations and ratios. Rather than an impracticably long and expensive process of trying all possible ratios and combinations of antioxidants present in a food-stuff, the antioxidants are tested in combinations and at ratios in which they occur in the food-stuff. In this way, combinations that are more likely to have synergistic antioxidant capacity will be tested.

An aspect is a method of manufacturing a nutritional-supplement with synergistic antioxidant capacity. In a food-stuff at least two antioxidant compounds are identified in a food-stuff, and their individual antioxidant capacity are determined. In addition, their ratio to each other in the food-stuff, the food-stuff ratio, is determined. By determining if the antioxidant capacity of the mixture is larger than the additive or expected capacity, which is sum of the antioxidant capacities of the compounds in the mixture, taken individually, it can be determined whether there is synergy between the compounds in the antioxidant capacity.

An antioxidant compound is a compound having antioxidant capacity. Any suitable system can be used to measure antioxidant capacity. In the examples, antioxidant capacity of single compounds and mixtures is determined by the Oxygen Radical Absorbance Capacity (ORAC) assay. It was selected among many choices of antioxidant assays for its common use and familiarity outside of academic research, as one of the goals was to show the potential application of the results either for human nutrition or food preservation. However, any suitable method for determining antioxidant capacity is contemplated. Suitable methods include, but are not limited to;

ORAC—Oxygen Radical Absorbance Capacity assay,
NORAC—Peroxynitrite ORAC assay,
HORAC—Hydroxyl ORAC assay,
ORAC-PG—Oxygen Radical Absorbance Capacity pyrogallol red assay,
DPPH—2,2-diphenyl-1-picrylhydrazyl radical assay,
FRAP—Ferric Reducing Ability of Plasma assay,
TEAC—Trolox Equivalent Antioxidant Capacity assay,
VCEAC—Vitamin C Equivalent Antioxidant Capacity assay,
ABTS—2′-azinobis-(3-ethylbenzothiazoline-6-sulfonic acid) assay,
CUPRAC—Cupric Reducing Antioxidant Capacity assay,
TRAP—Total Radical Trapping Antioxidant Parameter assay, and
CAA—Cellular Antioxidant Activity assay.

Synergism in antioxidant mixtures is determined by first forming a mixture comprising at least two of the antioxidant compounds at the foodstuff ratio, which is the ratio of the compounds in the foodstuff to each other, and determining antioxidant capacity of the mixture.

Synergism is determined by comparing the antioxidant capacity of the mixture with the expected or additive antioxidant capacity value. The additive value is the combined antioxidant capacity of each of the individual antioxidant compounds of the mixture, taken individually or assuming that each is functioning independently. The comparison can be calculated by subtracting the sum of the antioxidant capacities for the individual compounds from the resulting antioxidant capacity of the mixture of all the antioxidant compounds. A positive result indicates a synergism. A negative or statistically small positive or no value indicates antagonism or no interaction between the compounds. In making the measurements of the antioxidant capacity, the average of several samples will give a statistically better value.

Another aspect is a nutritional supplement made by forming a mixture of compounds with synergistic antioxidant capacity, which is a mixture of certain antioxidant compounds at ratios to one another that has been determined to have synergistic antioxidant properties.

It has been found that by starting with the individual phenolic antioxidants at the concentration ratios found in a specific foodstuff, such as a fruit, that synergism can be demonstrated using only endointeractions. This helps to explain the antioxidant capacity difference between whole food stuff and individual components, and also establish a base for the development of optimized fruit-derived antioxidant preservatives.

A foodstuff includes any food of plant origin grown for human consumption, including foods that have been subject to post processing, such as drying, freezing, heating (including pasteurization), mixing with other ingredients, or any processing applied to the food before being made available for human consumption. Any food containing phenolic antioxidant compounds is contemplated as a foodstuff and can be analyzed to determine synergistic combinations of antioxidant compounds. Examples include fruits (such as oranges, strawberries, and blueberries exemplified below), vegetables, nuts, eggs, vegetable oils, grains (including black rice), soy, chocolate, cinnamon, oregano, fermented drinks (red wine) tea and coffee. Certain meats include antioxidants, such as poultry and fish, and can be considered foodstuffs for determination of synergistic antioxidant ratios.

### DESCRIPTION OF DRAWINGS

FIG. 1—Oxygen radical absorbance capacity (ORAC) differences for combinations minus the individual compounds in the combination (Eq. 1 to Eq. 3). All combinations shown are statistically significant (p<0.05 using Fisher's least significant difference); combinations that were not statistically significant are not shown. C=chlorogenic acid; H=hesperidin; L=luteolin; M=myricetin; N=naringenin; P=p-coumaric acid; Q=quercetin. HC indicates the ORAC of the mixture of H and C minus the ORAC of H and the ORAC of C, likewise for the other combinations. Each value is the mean of 4 replications.

FIG. 2—Oxygen radical absorbance capacity (ORAC) of combinations of 3 phenolic compounds at the concentration found in oranges minus the sum of the 2+1 ORAC data (Eq. 4). Analysis of the data in this way elucidates patterns and makes it possible to determine which compound interactions are most influential on the ORAC (see text for further discussion). All combinations shown are statistically significant (p<0.05 using ANOVA estimates); combinations that were not statistically significant are not shown. C=chlorogenic acid; H=hesperidin; L=luteolin; M=myricetin; N=naringenin; P=p-coumaric acid; Q=quercetin. HC+N indicates the ORAC of the mixture of H, C, and N minus the ORAC of the mixture of HC and the ORAC of N, likewise for the other combinations. Each value is the mean of 4 replications.

FIG. 3—Oxygen radical absorbance capacity (ORAC) of combinations of 4 phenolic compounds at the concentration found in oranges minus the sum of the 3+1 ORAC data (Eq. 5). Analysis of the data in this way elucidates patterns and makes it possible to determine which compound interactions are most influential on the ORAC (see text for further discussion). All combinations shown are statistically significant (p<0.05 using ANOVA estimates); combinations that were not statistically significant are not shown. C=chlorogenic acid; H=hesperidin; L=luteolin; M=myricetin; N=naringenin; P=p-coumaric acid; Q=quercetin. HC+N indicates the ORAC of the mixture of H, C, and N minus the ORAC of the mixture of HC and the ORAC of N, likewise for the other combinations. Each value is the mean of 4 replications.

FIG. 4—Structures of phenolic compounds and their one-electron reductions potentials.

FIG. 5—Structures of antioxidant compounds in strawberries.

FIG. 6—ORAC of individual compounds in blueberries.

FIG. 7—ORAC of 1:1 ratio mixtures and fruit-ratio mixtures compared with expected results.

### DETAILED DESCRIPTION

### Example 1

### Synergistic and Antagonistic Interactions of Phenolic Compounds Found in Navel Oranges

Interactions of individual phenolic compounds (chlorogenic acid, hesperidin, luteolin, myricetin, naringenin, p-coumaric acid, and quercetin) at the concentrations found in navel oranges (Citrus sinensis) were analyzed for their antioxidant capacity to observe potential antagonistic, additive, or synergistic interactions. Mixtures of 2, 3, and 4 phenolic compounds were prepared. The Oxygen Radical Absorbance Capacity (ORAC) assay was used to quantify the antioxidant capacities of these combinations. Three different combinations of 2 compounds and 5 combinations of 3 compounds were found to be synergistic. One antagonistic combination of 2 was also found. No additional synergism occurred with the addition of a 4th compound. A model was developed to explain the results. Reduction potentials, relative concentration, and the presence or absence of catechol (o-dihydroxy benzene) groups were factors in the model.

Materials and Methods

Chemicals

Trolox ((±)-6-Hydroxy-2,5,7,8-tetramethylchromane-2-carboxylic acid) (97% purity, Acros Organics), naringenin (95%, MP Biomedicals Inc.), quercetin hydrate (95%, Acros Organics), sodium hydroxide (50% solution), K₂HPO₄, and KH₂PO₄ (Mallinckrodt Inc.) were purchased through Fisher Scientific Inc. (Waltham, Mass., U.S.A.). Chlorogenic acid (95%), hesperidin (>80%), luteolin (99%), myricetin (95%), p-coumaric acid (98%), and fluorescein (Na salt) were purchased from Sigma-Aldrich (St. Louis, Mo., U.S.A.). AAPH (2,2′-Azobis(2-methylpropionamidine) dihydrochloride) was purchased from Wako Chemicals U.S.A. Inc. (Richmond, Va., U.S.A.).

Chemical Preparation

Seven of the most concentrated phenolics found in oranges were selected: chlorogenic acid, hesperidin, luteolin, myricetin, naringenin, p-coumaric acid, and quercetin (Proteggente and others 2003; Franke and others 2004; USDA Flavonoid Database 2007). Each was quantified in the cited references as aglycones, with the exception of hesperidin. All compounds were prepared at the published concentrations (Table 1).

TABLE 1
Selected phenolic compounds and the
amount found in navel oranges.

| Compound | Mg/100 g fresh fruit |
|---|---|
| Chlorogenic acid | 0.19 |
| Hesperidin | 31 |
| Luteolin | 0.7 |
| Myricetin | 0.01 |
| Naringenin | 7.1 |
| P-courmaric acid | 0.02 |
| Quercetin | 0.2 |

Because of the high water content of oranges, no density adjustment was made. Compounds were prepared assuming 100 g was 100 mL in volume. All compounds except hesperidin and luteolin were weighed (10× to 1000× of Table 1 concentration to facilitate weighing) and dissolved in methanol. Luteolin and hesperidin were prepared in an 8:2 (v:v) mixture of methanol and 1N NaOH at room temperature (RT), as these two compounds were only fully soluble at weighable concentrations in a basic solution. The phenolic stock solutions were stored in 1 mL aliquots at −20.C. Phenolics were brought to RT, vortexed, and diluted in 7:3 (v:v) acetone:water to match the fruit concentrations in Table 1. To fit the Trolox standard curve (see below for assay description), compounds were further diluted in 7:3 (v:v) acetone:water to the following molar concentrations prior to transfer to the 96-well plate: chlorogenic acid, 10.7 μM; hesperidin, 10.2 μM; luteolin, 2.45 μM; myricetin, 0.786 μM; naringenin, 2.61 μM; pcoumaric acid, 1.95 μM; and quercetin, 6.62 μM. Solubility was checked after thawing and dilution. All work involving phenolic compounds, fluorescein, and Trolox was performed in dark conditions to minimize degradation.

Mixtures

All possible combinations of 2 compounds were mixed on an equal volume basis after being prepared at the concentrations found in Table 1 to ensure relative concentrations were maintained. Mixtures were then further diluted to match the lowest individual compound molarity to fit the Trolox standard curve. After determining the ORAC and completing statistical analyses, the top 3 statistically synergistic combinations of 2 were combined with all possible 3rd compounds and likewise analyzed. The same pattern was repeated for combinations of 4: the top 3 synergistic combinations of 3 were combined with all possible 4th compounds. Combinations of 2, 3, and 4 compounds were prepared on the same day of their ORAC assay.

Oxygen Radical Absorbance Capacity (ORAC)

The ORAC assay was performed according to Davalos and others (2004) with some modifications. Briefly, fluorescein was diluted in phosphate buffer to 70.3 mM and stored in 25 mL aliquots for not more than a month at −20 degrees C. Trolox was diluted to 80 μM in a 7:3 mixture of acetone and water, and stored at −20 degrees C. in aliquots of 100 μL for not more than a month. AAPH was diluted to 12 mM in phosphate buffer 5 minutes prior to each ORAC assay. Fluorescein and AAPH were heated to 37 degrees.C and transferred to all wells of 96-well plates via a Precision Micropipettor (BioTek Instruments Inc., Winooski, Vt., U.S.A.). All concentrations of Trolox (10 μM, 20 μM, 40 μM, 60 μM, 80 μM) were transferred in duplicate wells within the same row to form a standard curve. Phenolic solutions were transferred to wells in duplicate according to a predesigned plate layout. All filled plates were warmed within the plate reader (set at 37 degrees C.) for 15 min prior to the addition of AAPH and subsequent fluorescence measurement. Each mirrored duplicate was averaged and counted as 1 replicate. All samples were measured in quadruplicate (8 wells total) to obtain necessary statistical power.

Fluorescence of all wells was measured at 485/20 nm excitation and 528/20 nm emission every minute for 120 min in a BioTek Synergy 2 fluorescence plate reader (BioTek Instruments Inc.). ORAC values were expressed as Trolox Equivalents per liter (TE/L) of solvent containing the concentration of phenolic(s) found in navel oranges.

Statistics

For combinations of two, a difference was calculated by subtracting the sum of the average ORAC values for the individual compounds from the resulting average ORAC value of the combination of both compounds (Eq. 1):

Difference=(combination ab)−(individual a+individual b). (1)

Likewise, for combinations of 3 and 4, the difference was calculated by subtracting the average of the individual 3 or 4 compounds from the combination (Eqs. 2 and 3).

Difference=(combination abc)−(a+b+c), (2)

Difference=(combination abcd)−(a+b+c+d). (3)

Presenting the results in this manner allowed one to easily distinguish those combinations that were at minimum additive, using Fisher's least significant difference (LSD) analysis in the SAS statistical package (SAS Institute Inc., Cary, N.C., U.S.A.).

Additionally, for combinations of 3 and 4, a difference was calculated by subtracting the sum of the average ORAC values for the combination of 2 or 3, plus 1 individual, from the resulting average ORAC value of the combination of all 3 or 4 compounds (Eqs. 4 and 5).

Difference=(combination abc)−(combination ab+individual c) (4)

Difference=(combination abcd)−(combination abc+d) (5)

SAS was used to determine significance of combinations using estimate statistics, which take into account error terms when data are combined. The above described differences were compared through an ANOVA of the individual and combination results of the ORAC values, and forming the differences as post hoc tests to determine the effect of combining the individual compounds and combinations.

Results and Discussion

Combination ORAC Minus the Sum of Individual Phenolic ORAC Values

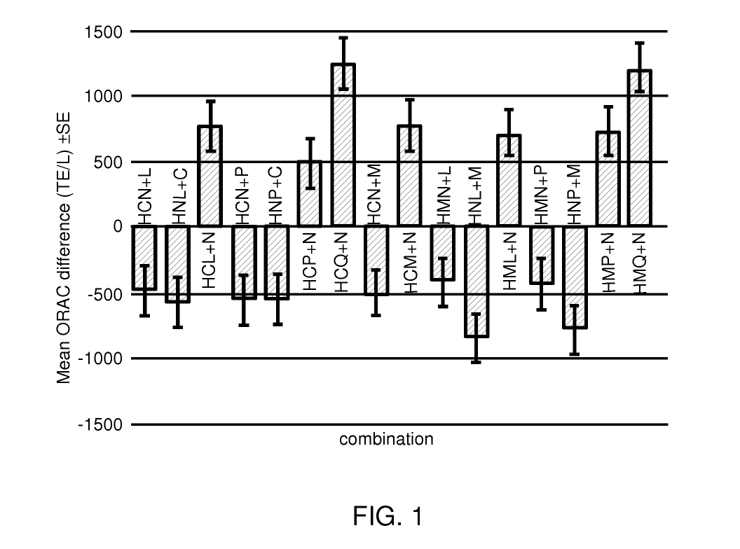

FIG. 1 presents ORAC values for all statistically significant combinations, as per Eq. 1 to Eq. 3. The combinations hesperidin/myricetin, hesperidin/naringenin, and hesperidin/chlorogenic acid had statistically synergistic ORAC values among the 21 two-way combinations tested. The combinations of 3 that showed significant differences were hesperidin/chlorogenic acid/naringenin, hesperidin/myricetin/naringenin, hesperidin/naringenin/luteolin, hesperidin/naringenin/p-coumaric acid, and hesperidin/naringenin/quercetin. The ORAC values of combinations of 4 were all significantly synergistic when the 4 individual values were subtracted.

Stepwise Analysis

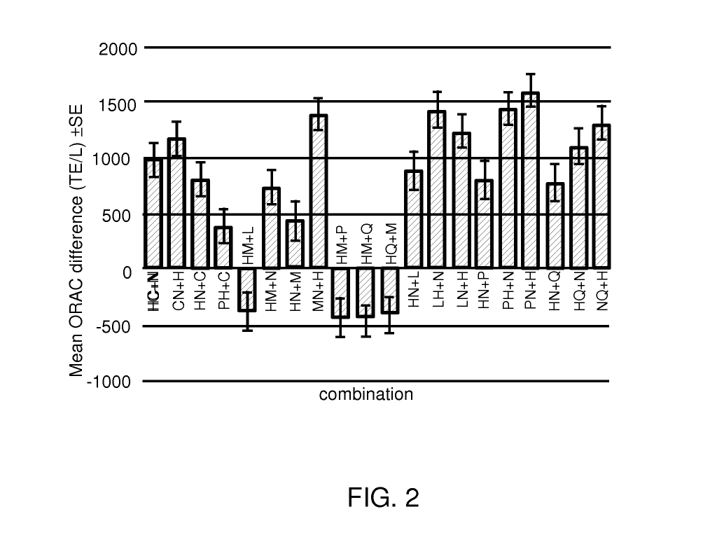

When analyzed in a stepwise manner (Eq. 4), values of some combinations of 3 were significant (FIG. 2). For example, hesperidin/chlorogenic acid+naringenin, chlorogenic acid/naringenin+hesperidin, and hesperidin/naringenin+chlorogenic acid were all significantly synergistic, all of which agree with the significant result for hesperidin/chlorogenic acid/naringenin in FIG. 1. Additionally, we found that combining hesperidin/naringenin or adding any 3rd compound to hesperidin/naringenin was always significantly positive. In other words, one other compound appears to increase hesperidin/naringenin's ORAC.

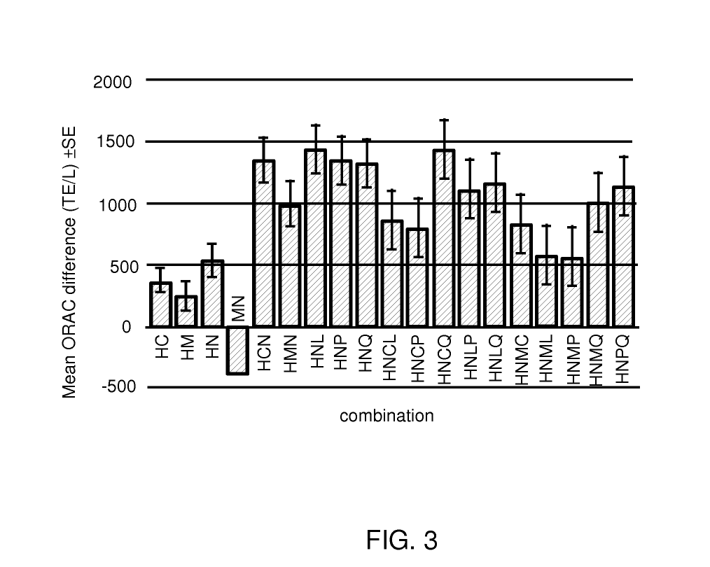

Despite the apparently positive results shown in FIG. 1, analysis of combinations of 4 (Eq. 5) showed that combinations of 4 did not have significantly higher raw ORAC values than the combinations of 3 (compare FIGS. 1 and 3). Additionally, if the combination already included hesperidin and naringenin, adding a 4th compound nearly always decreased the ORAC. Adding naringenin to any combination containing hesperidin always significantly increased the ORAC, as found in combinations of 2 and 3. In no case where hesperidin and naringenin were already together did adding a 4th compound increase the ORAC. A 4th compound appears to decrease hesperidin/naringenin's performance as an antioxidant pair or does not affect it. In contrast to additive combinations (Eq. 1, FIG. 1), the combinations of 2+1 and 3+1 that had a significantly lower ORAC value than the sum of individual phenolics, predominantly contained myricetin or p-coumaric acid.

Antagonistic Interactions

Antagonistic interactions were apparent in several of the combinations. The only combination of 2 to show significant antagonism was myricetin/naringenin. No combinations of 3 or 4 in the additive analysis (Eq. 2 and Eq. 3) were significantly antagonistic. In the stepwise analysis of (Eq. 4 and Eq. 5), there were several statistically significant antagonistic interactions (see FIGS. 2 and 3). Myricetin was part of all 2+1 combinations that showed antagonistic interactions. The addition of hesperidin to the antagonistic combination of myricetin/naringenin removed the antagonism in the combination, instead resulting in strong synergism. Myricetin is also present in 5 of the 3+1 combination antagonistic interactions, though there are no apparent patterns in the other four 3+1 combinations that were antagonistic.

Combinations of 5 or More

Overall, we found that several combinations of 2, 3, and 4 compounds demonstrated significant synergism when combined. On the basis of those results and the observed interactions, we predicted that greater complexity would not have significantly higher antioxidant capacity than that already achieved in combinations of 3. The increase in complexity of combinations past the level of 3 compounds did not increase the total ORAC of the combination (FIG. 1). There were no further interactions found with combinations of 4 that were not already occurring with combinations of 3. Thus, no further analyses of combinations of 5 or more were performed.

Structural Analysis

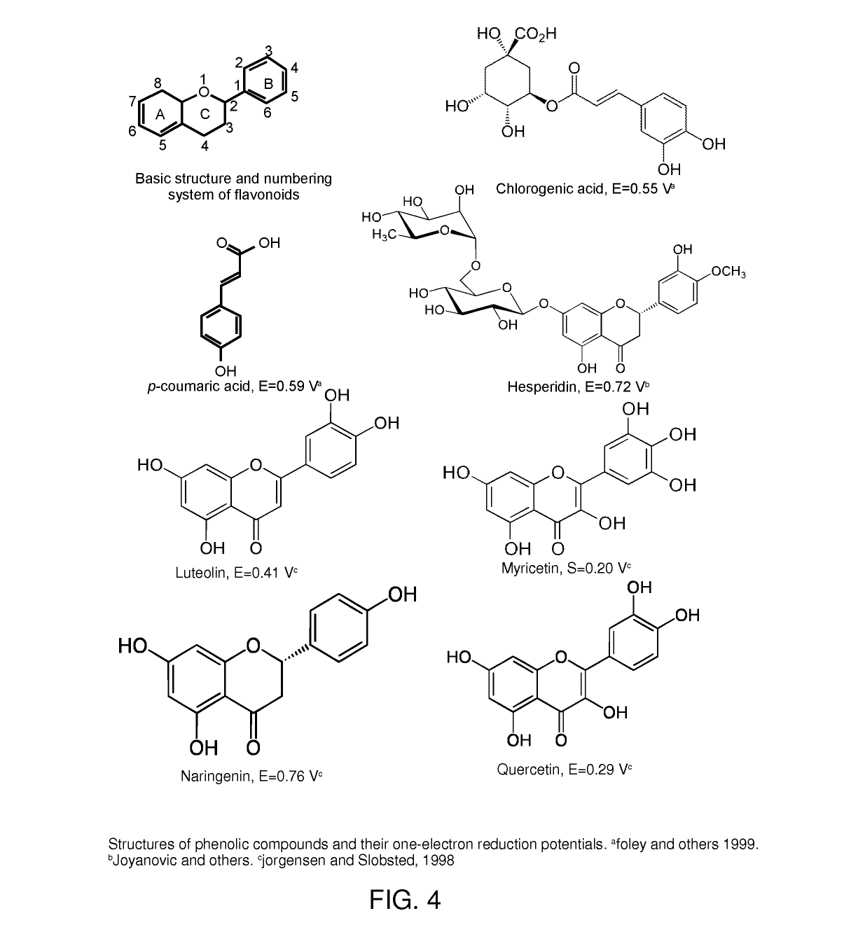

While not being bound to any theory, it is believed that the antioxidant capacity of phenolic compounds is dependent on the arrangement and number of hydroxyl groups on the ring structure, with a catechol group in the B ring and 2, 3 double bonds in the C ring (see FIG. 4) being 2 characteristics that have been shown to strongly correlate with antioxidant capacity (Rice-Evans 2001; Ami'c and others 2007). These 2 functional groups also predict reduction potentials, which will be discussed antagonism. Luteolin also has a catechol group in the B ring and later. Based on these functional groups, we made the following a 2, 3 double bond in the C ring, and shows results similar to observations from these results: Myricetin has both a catechol group myricetin. On the other hand, the 2 compounds that showed the in its B ring and a 2, 3 double bond in its C ring. However, it did strongest synergism do not have structural characteristics related not show a strong relationship in improving antioxidant capacity to antioxidant strength. Both naringenin and hesperidin do not in these experiments. In fact, this compound showed significant have catechol groups or 2, 3 double bonds, yet are the compounds present in all combinations that showed synergism. Furthermore, hesperidin is a glycoside, which has been shown to further hinder the molecule's antioxidant capacity (Di Majo and others 2005). Naringenin and hesperidin are the 2 compounds with the highest concentration and closest molar ratio in these combinations, which may explain their apparent synergism (Cuvelier and others 2000).

Several hypotheses have been developed to explain synergistic and antagonistic effects of antioxidant combinations. Peyrat-Maillard and others (2003) described combinations of a weak antioxidant with a strong antioxidant, where the weak antioxidant may regenerate the strong antioxidant, thus improving overall radical quenching ability of the combination. In a similar situation, antagonism may be explained by the strong antioxidant regenerating the weak antioxidant, which in turn quenches the radical. This would decrease the overall antioxidant strength of the combination. In a combination of a strong antioxidant with another strong antioxidant, the 2 compounds may regenerate each other and thus improve antioxidant strength overall. Other postulates given to explain the interactions of antioxidants include the reaction rates of the antioxidants, the polarity of the interacting molecules, and the effective concentration of the antioxidants at the site of oxidation (Frankel and others 1994; Koga and Terao 1995, Cuvelier and others 2000).

Reduction Potentials

While not being bound to any theory, expected interactions can also be theoretically determined by using one-electron reduction potentials of phenolic antioxidants (FIG. 4). The lower the reduction potential, the more likely the molecule is to donate its electrons. It is also more likely to donate its electrons to the molecule with the next highest E value. This adds a quantitative basis to the explanation provided by Peyrat-Maillard and others (2003). Based on these reduction potentials (Jovanovic and others 1994; Foley and others 1999; Jorgensen and Skibsted, 1998), the 7 compounds used can be ordered as follows: myricetin>quercetin>luteolin>chlorogenic acid>p-coumaric acid>hesperidin>naringenin. Add the peroxyl radicals generated by AAPH (E=approximately 1 V; Buettner 1993) after naringenin. This would suggest that, at equimolar concentrations, myricetin would always donate its electrons to (recycle) quercetin, then luteolin, and so forth to the peroxyl radical. However, in the case of navel oranges, there are significant differences in relative concentrations. Hesperidin and naringenin, which have the highest reduction potentials, are also found at significantly higher relative concentrations than the other 5 phenolic compounds analyzed.

Theoretically, all combinations of 2 could be synergistic if one of the 2 species donates its electrons to the other, allowing it to more effectively scavenge the peroxyl radicals produced by AAPH. The hierarchy of donation is also clear based on the reduction potentials. For example, in the combination of hesperidin and naringenin, hesperidin will donate electrons to naringenin, which will donate to the peroxyl radical. However, this does not result in accurate predictions. Only a few combinations were significant; not all.

Reduction potentials are a measure of single electron transfer (SET), while the ORAC assay reaction mechanism is based on hydrogen atom transfer (HAT). Unfortunately, there are no volt measures of HAT available for phenolic compounds. However, the end result is still the same (Ou and others 2002). In both SET and HAT, a peroxyl radical ultimately becomes peroxide, and the antioxidant loses an electron, with a resulting weakly reactive unpaired electron in its structure. An electron must be abstracted in both mechanisms. Order of phenolic reactivity can, thus, be assumed to be similar between the 2 mechanisms. This assumption was made in order to develop a model with a quantitative basis.

A Model

While not being bound to any theory, by focusing on the presence or absence of a catechol group (or methoxy catechol group on hesperidin), the reduction potential and the relative concentration, the synergistic (and antagonistic) combinations of 2 can be explained. The phenolic molecules with a catechol group have lower reduction potentials and will donate their electrons more readily. If there is a molecule at a lower relative concentration with a catechol group that is in a combination with a molecule without a catechol group, the electron donation is minimized. This is the case with myricetin/naringenin. However, with myricetin/hesperidin, the donation is more efficient, producing synergy (likewise for hesperidin/chlorogenic acid) due to the methoxy catechol group on hesperidin, which is better recycled than a compound with a single hydroxyl group on the B ring. In the case of hesperidin/naringenin, even though the donation is inefficient (from a catechol to a noncatechol), concentration overpowers (hesperidin is present at 4× the concentration of naringenin), and the combination is significant.

There are a few combinations that do not fit this model. Myricetin/quercetin, luteolin/quercetin, and myricetin/luteolin all had simply additive ORAC, though each has a catechol group that could theoretically donate to its combination pair. Similarity of structure may make interaction and donation of electrons to each other inefficient, as they may simply donate back and forth to some extent, resulting in an additive-only ORAC. The compounds in these combinations appear to interact independently, or additively, with the peroxyl radicals until they are destroyed (ring structure cleaved).

The same model also applies to combinations of 3. All combinations that were significant included hesperidin and naringenin, though the addition of a 3rd compound increased the magnitude of the ORAC difference (FIG. 1). The addition of a third compound with a lower reduction potential, despite its very low concentration compared with hesperidin or naringenin, increased the efficiency of electron transfer or preservation of them sufficiently to add magnitude to the resulting ORAC value. When comparing hesperidin/naringenin+a 3rd compound (FIG. 2), the order of benefit is luteolin>quercetin=chlorogenic acid=p-coumaric acid>myricetin at increasing the ORAC, which is similar to the concentration (Table 1), though not the reduction potential order discussed above (myricetin>quercetin>luteolin>chlorogenic acid>p-coumaric acid). In this case, concentration is more important than functional groups or efficiency of electron donation.

For combinations of 4, the addition of a 4th compound (FIG. 3) decreased the efficiency of many combinations, with synergism only present in those combinations that added naringenin to a combinations containing hesperidin. In cases where hesperidin and naringenin were already in a group of 3, the addition of a 4th compound had no effect or was antagonistic. They do not appear to fit the catechol group/reduction potential/concentration model described above. The magnitudes of the significantly antagonistic results were all small compared to the magnitudes of the synergistic results in combinations of 3, 4, 2+1, and 3+1. It is likely that the 4th compound decreases the efficiency of electron transfer between strong groups of 3. This would account for all of the antagonistic combinations.

Conclusion of this Example

Our hypothesis that synergistic interactions would occur between phenolic compounds at the concentrations and ratios found in navel oranges was found to be true. The interaction between naringenin and hesperidin provided the most synergism, while the addition of a 3rd compound enhanced that synergism. Addition of a 4th compound did not significantly add to the magnitude of the ORAC compared to combinations of 3. Analyzing together (1) functional groups, (2) reduction potentials, and (3) relative concentration best explained the synergistic and antagonistic interactions. These synergistic phenolic interactions have the potential application of preserving food or beverages.

### Example 2

### Synergistic Phytochemical Combinations Found in Oranges

Supplement composition were prepared based upon data derived from procedures as illustrated in Example 1.

In Table 2 is shown strong combinations of phytochemicals found in navel oranges. Also, for comparison, included are two products currently marketed for their high ORAC values. The table is ordered from highest ORAC per gram to lowest.

The most promising combination in the table is hesperidin/naringenin/p-coumaric acid/quercetin, as they demonstrated 29% synergy together and are all readily available at low costs, as shown in Table 3.

The combinations that show synergism have the potential to make a significant improvement in the quality and antioxidant power of supplements. Rather than simply combining individual fruits at random or creating concentrated extracts with unknown toxicity, the data demonstrates the power that rations that fruit provide, while providing a very effective and safe dose.

For example: Using 1 gram of antioxidant mixture in a supplement would be the equivalent of about 3000 g, or 6 lbs, of oranges. This would be unrealistic to consume and perhaps unsafe. A capsule containing around a third of this would conservatively represent an amount of fruit that could be consumed in a day, ensuring the safety of such a quantity, while still providing exceptional synergistic antioxidant protection. A capsule would also provide convenience, more antioxidant than one could reasonably consume in the form of fruit, a long-term shelf life, and a company to stand behind the product.

TABLE 2

| | ORAC Value | |
|---|---|---|
| | (μmol Trolox | Synergy (% |
| | Equivalents/g | increase over sum |
| Combination/Product Name | of mixture) | of individuals) |
| Hesperidin/naringenin/luteolin | 14327 | 35% |
| Hesperidin/naringenin/ | 14048 | 37% |
| chlorogenic acid/quercetin | | |
| Nature's Answer OR AC Super 7 | 13,917 | N/A |
| Hesperidin/naringenin/ | 13903 | 29% |
| p-coumaric acid/quercetin | | |
| Hesperidin/naringenin/ | 13817 | 35% |
| p-coumaric acid | | |
| Hesperidin/naringenin/ | 13777 | 34% |
| quercetin | | |
| Hesperidin/chlorogenic acid/ | 13753 | 35% |
| naringenin | | |
| Hesperidin/naringenin/ | 13487 | 27% |
| luteolin/p-coumaric acid | | |
| Hesperidin/naringenin/ | 13008 | 26% |
| myricetin/quercetin | | |
| Hesperidin/naringenin/ | 12983 | 28% |
| luteolin/quercetin | | |
| Hesperidin/myricetin/ | 12918 | 26% |
| naringenin | | |
| Hesperidin/naringenin | 11648 | 14% |
| Hesperidin/chlorogenic acid | 8316 | 16% |
| Hesperidin/myricetin | 8009 | 11% |
| Future Biotics Antioxidant | 4,583 | N/A |
| Superfood | | |
| cinnamon | 2,640 | N/A |
| ascorbic acid (vitamin C) | 2,000 | N/A |
| Navel oranges | 18 | N/A |

TABLE 3
Current retail costs from chemical supplier Sigma:

| compound | $ | Amount | | per mg |
|---|---|---|---|---|
| Chlorogenic acid | 281.50 | 5 | g | 0.0563 |
| Hesperidin | 127.50 | 100 | g | 0.00128 |
| Luteolin | 281.50 | 50 | mg | 5.63 |
| Myricetin | 293.00 | 100 | mg | 2.93 |
| naringenin | 161.50 | 25 | g | 0.00646 |
| p-coumaric acid | 68.5 | 25 | g | 0.00274 |
| quercetin | 155 | 100 | g | 0.00155 |

### Example 3

### Synergistic and Antagonistic Interactions of Phenolic Compounds Found in Strawberries

The interactions of mostly aglycones of seven phenolic compounds at relative concentrations found in strawberries were tested using the Oxygen Radical Absorbance Capacity (ORAC) assay. Interactions that occurred in simpler combinations were explored in more complex combinations. A model was developed to explain why the interactions occurred. Statistically significant synergism was observed among three combinations of two phenolic compounds, and among five combinations of three phenolic compounds. Statistically significant antagonism was observed among two combinations of two phenolic compounds and among one combination of three compounds. A model that includes reduction potentials, relative concentration, and the presence or absence of catechol (o-dihydroxy benzene) groups explains the results. This example demonstrates some of the interactions that can occur in a complex environment within the framework of strawberry phenolic compounds. The synergism found for food-based antioxidant ratios suggests strawberries have optimized free radical protection; this could be applied to food preservation.

Plants produce phenolic compounds to act as cell signaling molecules, antioxidants or poisons to invading pests (Crozier et al., 2006). A variety of these phenolic compounds are present in fruit and they have been widely characterized (Robards et al., 1999; Franke et al., 2004; Harnly et al., 2006). This characterization developed in part due to the high antioxidant capacity of these compounds.

Strawberries are a good source of phenolic compounds (Aaby et al., 2005), with a total phenolic content of about 290 mg gallic acid equivalents per 100 g fresh weight. They contain a wide variety of phenolic compounds, including cyanidin and pelargonidin glycosides, ellagic acid (including glycoside and tannin forms), catechin, procyanidins, cinnamic acid derivatives and flavonols. The oxygen radical absorbance capacity (ORAC) of raw strawberries is 35 μmol tocopherol equivalents (TE) per g fresh weight (2007 USDA ORAC database), which is lower than blueberries and raspberries, but higher than oranges or bananas.

It was hypothesized that by preparing individual phenolic antioxidants at the concentration found in strawberries (using aglycones in most cases), that combinations could be found with demonstrated synergism within the context of a strawberry. By using mostly aglycones, previously studied structural elements of flavonoids could be examined for the development of a model explaining observed results. While this would limit extrapolation of the results to the real fruit, it would help establish a basis for the development of optimized fruit-derived antioxidant preservatives, as has been explored with extracts. Complex interactions between seven phenolic compounds found in strawberries were analyzed using oxygen radical absorbance capacity (ORAC) and a model was developed to explain the results.

Material and Methods

Chemicals

Cyanidin chloride (purity: 95%), p-coumaric acid (98%), (+)-catechin (96%), quercetin-3-glucoside (90%), kaempferol (96%), ellagic acid (96%), pelargonidin chloride (95%), and fluorescein disodium salt were obtained from Sigma Chemical Co (St. Louis, Mo., USA). Trolox (6-hydroxy-2,5,7,8-tetramethyl-2-carboxylic acid), sodium hydroxide (50% solution), K₂HPO₄ and KH₂PO₄ and Corning Costar 96-well black side clear bottom plates were obtained from Fischer Scientific (Pittsburgh, Pa., USA) and 2,2′-Azobis(2-amidinopropane) dihydrochloride (AAPH) was obtained from Wako Chemical USA (Richmond, Va., USA).

Chemical Preparation

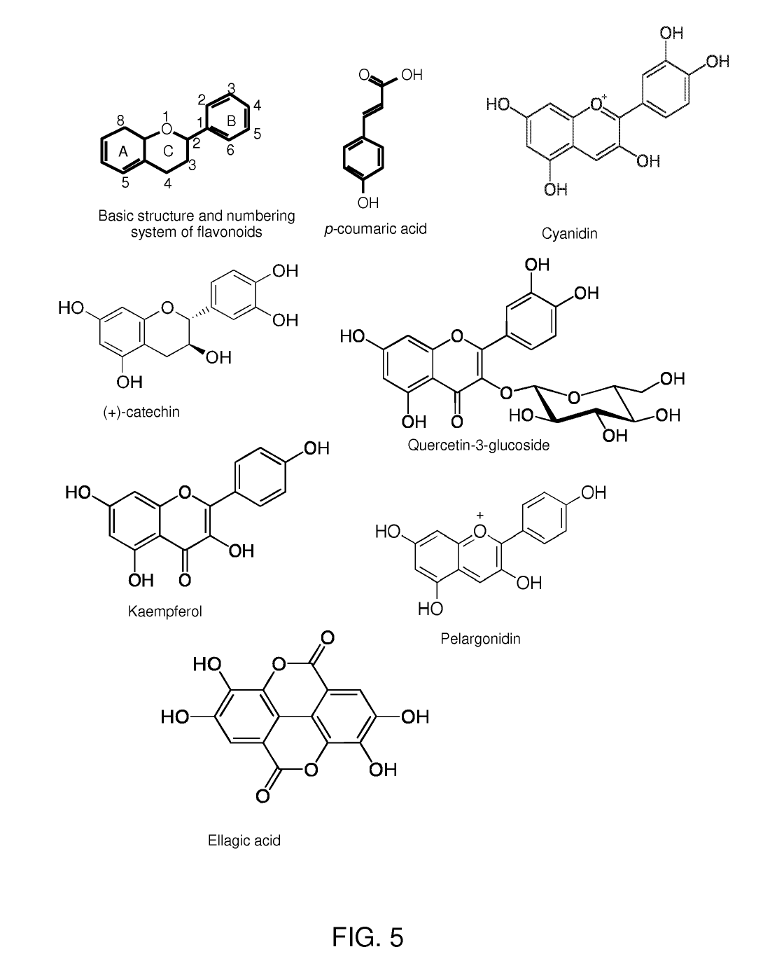

Table 4 shows the concentrations of the seven compounds studied as found in cultivated strawberries. FIG. 5 provides the structures. Compounds were selected based on highest average concentration in strawberries. Concentrations used were selected from previously published (2007 USDA flavonoid database; Zhao, 2007) absolute concentrations of strawberry phenolics, assuming complete hydrolysis of glycosides (though not tannins) to facilitate later modeling. It was assumed that 1 g fruit puree was 1 ml in volume for sample preparation, as a density adjustment would not change relative concentrations that would be tested. All compounds except ellagic acid were weighed then dissolved in methanol. Ellagic acid was weighed and dissolved in a heated 4:1 mixture of methanol and 1 M sodium hydroxide, as it was only fully soluble at weighable concentrations in a basic solution. The phenolic stock solutions were stored in 1 mL aliquots at −20° C. Phenolics were brought to RT, vortexed, and diluted in 7:3 (v:v) acetone:water to match the fruit concentrations in Table 4. To fit the Trolox standard curve (see below for assay description), compounds were further diluted in 7:3 (v:v) acetone:water to the following molar concentrations prior to transfer to the 96-well plate: p-coumaric acid, 9.99 μM; cyanidin, 3.04 μM; catechin, 4.58 μM; quercetin-3-glucoside, 2.45 μM; kaempferol, 1.61 μM; pelargonidin, 5.10 μM; ellagic acid, 15.4 μM. Solubility was checked after thawing and dilution. All work involving phenolic compounds, fluorescein, and Trolox was performed in dark conditions to minimize degradation.

TABLE 4
Concentrations of strawberry phenolic compounds studied.

| Compound | mg/100 g fresh weight^{a} |
|---|---|
| p-coumaric acid | 4.10^{b} |
| cyanidin | 1.96^{c} |
| (+)-catechin | 3.32^{c} |
| quercetin-3-glucoside | 1.14^{d} |
| kaempferol | 0.46^{c} |
| pelargonidin | 31.3^{c} |
| ellagic acid | 46.5^{d} |

^{a}No density adjustment was made; compounds were prepared assuming 100 g was 100 ml in volume, as the ORAC analyses assessed relative ratios only.
^{b}Maximum amount reported by Zhao (2007).
^{c}2007 USDA Flavonoid database.
^{d}Average value reported by 2007 USDA flavonoid database and Zhao (2007).

Mixtures

All possible combinations of two compounds were mixed on an equal volume basis after being prepared at the concentrations found in Table 4 to ensure relative concentrations were maintained. Mixtures were then further diluted to match the lowest individual compound molarity to fit the Trolox standard curve. After determining the ORAC and completing statistical analyses, the top three statistically synergistic combinations of two were combined with all possible third compounds and likewise analyzed. The same pattern was repeated for combinations of four: the top three synergistic combinations of three were combined with all possible fourth compounds. No statistically significant increases in antioxidant capacity were found for combinations of four. Thus combinations of 5 or 6 were not tested, though all seven compounds in combination were assayed. Combinations were prepared on the same day of their ORAC assay.

Oxygen Radical Absorbance Capacity (ORAC) Assay

ORAC assays were carried out according to Dávalos et al. (2004), with some modifications, using a Biotek Synergy 2 plate reader (BioTek Instruments, Inc., Winooski, Vt., USA). The reaction was performed in 75 mM phosphate buffer (pH 7.1) and the final assay mixture (200 μl) contained fluorescein (120 μl, 70.3 nM final concentration) as oxidizable substrate, AAPH (60 μl, 12 mM final concentration) as oxygen radical generator, and antioxidant (20 μl, either Trolox [1-8 μM, final concentration] or sample). Parameters of the assay were as follows: reader temperature: 37 degrees C., cycle number, 120; cycle time, 60 seconds; shaking mode, 3 seconds of orbital shaking before each cycle. A fluorescence filter with an excitation wavelength of 485/20 nm and an emission wavelength of 520/20 nm was used. Samples were prepared in 96-well plates in a mirror fashion, based on a planned layout. Each mirrored duplicate was averaged and counted as one data point. All samples were measured in quadruplicate (eight wells total) to obtain necessary statistical power. Data are expressed as micromoles of Trolox equivalents (TE) per liter of solution. The data were analyzed using a Microsoft Excel 2007 (Microsoft, Redmond, Wash., USA) spreadsheet to determine area under the curve and to convert the data to Trolox equivalents based on the Trolox standard curve.

Statistics

For combinations of two, a difference was calculated by subtracting the sum of the average ORAC values for the individual compounds from the resulting average ORAC value of the combination of both compounds (equation 6).

Difference=(combination ab)−(individual a+individual b) (6)

Likewise, for combinations of three and four, the difference was calculated by subtracting the average of the individual three or four compounds from the combination. Presenting the results in this manner allowed us to easily distinguish whether the combination was greater or less than the sum of its parts, using mixed model ANOVA estimates in the Statistical Analysis Software statistical package (version 9.1, SAS Institute Inc., Cary, N.C., USA).

Additionally, for combinations of three and four, a difference was calculated by subtracting the sum of the average ORAC values for the combination of two or three, plus one individual, from the resulting average ORAC value of the combination of all three or four compounds (equations 7 and 8).

Difference=(combination abc)−(combination ab+individual c) (7)

Difference=(combination abcd)−(combination abc+d) (8)

SAS was used to determine significance of combinations using mixed model ANOVA estimates, which take into account error terms when data are combined. The above described differences were compared in SAS through an ANOVA of the individual and combination results of the ORAC values and forming the differences as post hoc tests to determine the effect of combining the individual compounds and combinations.

Results and Discussion for this Example

Compound and Combination Selections

The seven compounds we selected do not represent all phenolic compounds in strawberries, but a selection of those most highly concentrated and available commercially. The amounts we selected represent multiple studies (Aaby et al., 2005; 2007 USDA flavonoid database; Zhao, 2007) and a maximum quantity assuming complete hydrolysis of glycosides (though not tannins, in the case of ellagic acid, and excepting quercetin-3-glucoside). This does not necessarily reflect the total available for reaction in the digestive tract (Halliwell et al., 2000), as a significant portion of the phenolic compounds in a strawberry would be consumed as glycosides, and enzyme activity, digestive factors and other foods that may be present at the same time would impact the interactions. It is representative of average analytical amounts found in strawberries across many seasons and explored in multiple laboratories. The strawberry origin provides a framework for the chemistry being explored.

Evaluating the addition of one compound at a time to combinations allowed us to determine when to stop, i.e. since 3+1 combinations of four were no more significant than 2+1 combinations of three, we can predict that 4+1 combinations of five would also not be any more significant than combinations of three. To confirm this prediction, we tested the combination of all seven compounds together (11045±458 μmol TE/L). The magnitude of the ORAC value was no larger than that found with combinations of four.

Additive and Stepwise Analysis

The ORAC of statistically significant combinations of two are presented in Table 5. The statistical method was performed using equation 6 or its equivalent for all combinations of two, three and four; statistically significant results were only found for combinations of two, which are included in the figure. All other combinations of two, three or four were not significant and were considered additive. In the stepwise analysis (equations 7 and 8), all combinations of three and four were additive except those included in Table 5.

Phenolic Structure

Structure is an important determinant of antioxidant potential (Rice-Evans et al., 1996). The o-dihydroxy groups (catechol structure) in the B ring allows for greater stability to the radical form and participation in electron delocalization (FIG. 5). A 2, 3 double bond in conjugation with a 4-oxo in the C ring and 3- and 5-OH groups with a 4-oxo function in the A and C rings are essential for maximum radical quenching potential. Degree of hydroxylation is also important to antioxidant activity.

TABLE 5
Mean ORAC differences for statistically significant
combinations of phenolic compounds.^{a}

| | ORAC | Standard | |
|---|---|---|---|
| Combination^{b} | difference^{c} | Error | p-value^{d} |
| PcCa | 531 | 205 | 0.01 |
| PcQu | 521 | 239 | 0.03 |
| PcPe | −883 | 219 | <0.01 |
| CyQu | 513 | 239 | 0.03 |
| CyPe | −868 | 219 | <0.01 |
| PcCa + Pe | −976.6 | 282 | 0.001 |
| PcPe + Qu | 1333 | 305 | <.0001 |
| CyPe + Qu | 1027 | 305 | <.0001 |
| CyEl + Qu | 636.7 | 305 | 0.04 |
| QuEl + Pc | 849.4 | 299 | 0.01 |
| QuEl + Cy | 747.5 | 299 | 0.01 |

^{a}Combinations of two were calculated according to equation 6 for statistical analysis. Combinations of three were calculated according to equation 7. For simplicity, non-significant combinations were assumed to be additive and not included in the table.
^{b}Pc—p-coumaric acid, Cy—cyanidin, Ca—(+)-catechin, Qu—quercetin-3-glucoside, Ka—kaempferol, Pe—pelargonidin, El—ellagic acid.
^{c}Values were considered to be significant at p < 0.05 using mixed model ANOVA estimates.
^{d}Reported in μmol TE/L.

Several hypotheses have been developed to explain synergistic and antagonistic effects of antioxidant combinations. Peyrat-Maillard et al. (2003) suggested that along with other factors, some antioxidants in combination act in a regenerating manner, with either the stronger or weaker antioxidant regenerating the other. This can have an overall positive (synergistic) effect if the weaker antioxidant is regenerating the stronger antioxidant or an overall negative (antagonistic) effect if the opposite is occurring. Other postulates given to explain the interactions of antioxidants include the reaction rates of the antioxidants, the polarity of the interacting molecules and the effective concentration of the antioxidants at the site of oxidation (Frankel et al., 1994; Koga & Terao, 1995; Cuvelier et al., 2000).

Reduction Potentials

Expected interactions can also be theoretically determined used one-electron reduction potentials of phenolic antioxidants. The lower the reduction potential, the more likely the molecule is to donate its electrons. It is also more likely to donate its electrons to the molecule with the next highest E value. This adds a quantitative basis to the explanation provided by Peyrat-Maillard et al. (2003). Based on available published reduction potentials (Jorgensen & Skibsted, 1998; Foley et al., 1999), the seven compounds used can be ordered as follows: cyanidin>ellagic acid>quercetin-3-glucoside (0.29 V for quercetin; rutin, a diglucoside, is 0.4 V)>catechin (0.36 V)>pelargonidin>kaempferol (0.39 V)>p-coumaric acid (0.59 V). No published reduction potentials could be found for cyanidin, ellagic acid, or pelargonidin. They are ordered based on structural components that predict reduction potential.

Add the peroxyl radicals generated by AAPH (E=˜1 V; Buettner, 1993) after p-coumaric acid. This would suggest that, at equimolar concentrations, cyanidin would always donate its electrons to (recycle) ellagic acid, then quercetin-3-glucoside, and so forth to the peroxyl radical. However, using strawberry phenolic concentrations, there are significant differences in relative concentration. Ellagic acid and pelargonidin are found at significantly higher relative concentrations than the other five phenolic compounds analyzed.

Theoretically, all combinations of two could be synergistic if one of the two species donates its electrons to the other, allowing it to more effectively scavenge the peroxyl radicals produced by AAPH. The hierarchy of donation is also clear based on the reduction potentials. For example, in the combination of kaempferol and p-coumaric acid, kaempferol will donate electrons to p-coumaric acid, which will donate to the peroxyl radical. However, this does not result in accurate predictions. Only a few combinations were significant; not all.

Reduction potentials are a measure of single electron transfer (SET), while the ORAC assay reaction mechanism is based on hydrogen atom transfer (HAT). Unfortunately, there are no volt measures of HAT available for phenolic compounds. However, the end result is still the same (Ou et al., 2002). In both SET and HAT, a peroxyl radical ultimately becomes a peroxide, and the antioxidant loses an electron, with a resulting weakly reactive unpaired electron in its structure. An electron must be abstracted in both mechanisms. Order of phenolic reactivity can thus be assumed to be similar between the two mechanisms. This assumption was made in order to develop a model with a quantitative basis.

A Model

While not being bound to any particularly theory, it is believed that combining three factors, relative concentration, reduction potential, and the presence or absence of a catechol group, a model was developed to explain the results. Chosen phenolics were prepared in the following order of concentration:
ellagic acid>pelargonidin>p-coumaric acid>catechin>cyanidin>quercetin-3-glucoside>kaempferol (see Table 4).

One-electron reduction potentials place them in this order:
cyanidin≧ellagic acid>quercetin-3-glucoside>catechin>pelargonidin>kaempferol>p-coumaric acid.

Four of the seven compounds contain catechol groups:
ellagic acid, cyanidin, catechin, quercetin-3-glucoside

For those combinations of two (Table 4) that were statistically significant, p-coumaric acid was more concentrated than catechin. Catechin, with its catechol group and lower reduction potential, was a strong electron donor and helped recycle the more concentrated p-coumaric acid, producing synergy. p-coumaric acid and quercetin-3-glucoside interacted similarly. Cyanidin and quercetin-3-glucoside both contained catechol groups; cyanidin was present at a similar concentration, and both contained catechol groups, creating an environment for a synergistic result, likely with cyanidin recycling quercetin-3-glucoside (based on reduction potential). On the antagonistic side, p-coumaric acid combined with pelargonidin demonstrates the importance of the catechol group. Without it, pelargonidin is not an effective recycler of p-coumaric acid (which would be expected based on reduction potential), and with pelargonidin's much larger concentration, the presence of p-coumaric acid appears to disrupt pelargonidin's antioxidant activity, perhaps by drawing away electrons but not donating them as readily to the AAPH radical. This would suggest that pelargonidin's E value may be close to that of p-coumaric acid. Finally, cyanidin and pelargonidin also interacted antagonistically. Based on cyanidin's catechol group and reduction potential, synergism would be expected. Similarity of structure or relative concentration differences may explain the antagonism; this interaction does not fit this model, but persists in combinations of three and four. Again, the assumed E value order may be incorrect.

For combinations of three, no statistically synergistic or antagonistic results were found for additive combinations (per equation 6). However, when analyzed in a step-wise fashion (equation 7), significant results can be explained by the model described above. For quercetin-3-glucoside/ellagic acid+p-coumaric acid, two compounds with catechol groups and lower E values become more synergistic when p-coumaric acid is added. This is similar to what occurred with p-coumaric acid/(+)-catechin and p-coumaric acid/quercetin-3-glucoside. For quercetin-3-glucoside/ellagic acid+cyanidin and cyanidin/ellagic acid+quercetin-3-glucoside, the addition of another low E-value compound containing a catechol group enhanced the synergy of the combination. For p-coumaric acid/pelargonidin+quercetin-3-glucoside, the lower E-value quercetin-3-glucoside with its catechol group significantly improved the single hydroxyl group antioxidant efficiency of the other two compounds. And finally, for cyanidin/pelargonidin+quercetin-3-glucoside, the near doubling of available catechol groups (quercetin-3-glucoside and cyanidin have similar concentrations) gave a significant boost to the cyanidin/pelargonidin combination.

On the antagonistic side, one combination was significant: p-coumaric acid/catechin+pelargonidin. In this case, the significant synergism found with catechin donating electrons to p-coumaric acid (see Table 5) is disrupted by the large concentration of pelargonidin and its lack of a catechol group. This minimizes catechin's effectiveness and results in antagonism.

For combinations of four (data not shown), though no values were significant in either the additive or step-wise analyses, the trends follow the same pattern and are explained by the model. For example, p-coumaric acid/(+)-catechin/quercetin-3-glucoside+ellagic acid, p-coumaric acid/(+)-catechin/pelargonidin+quercetin-3-glucoside, and cyanidin/quercetin-3-glucoside/kaempferol+ellagic acid all had positive ORAC values, and all consisted of both catechol containing and non-catechol containing compounds that could donate electrons to each other in line with their reduction potentials. Two combinations had relatively high antagonistic ORAC values, p-coumaric acid/cyanidin/quercetin-3-glucoside+pelargonidin and p-coumaric acid/quercetin-3-glucoside/pelargonidin+cyanidin. In these cases, the higher relative concentration of catechol-lacking, lower reduction potential pelargonidin diminished the antioxidant capacity of these combinations.

Other Considerations

One potential concern is the effect of pH on anthocyanidins (Delgado-Vargas et al., 2000), two of which, cyanidin and pelargonidin, were included. Anthocyanidins are most stable at a pH of 2. As pH increases, anthocyanidins more readily react with water, losing their color and converting to chalcones. Light increases the degradation and the presence of other phenolic compounds slows the degradation of the anthocyanidins. In the present example, compounds were dissolved in methanol, so no water was present, all steps were performed in the dark, and when the solution was added to the aqueous ORAC mixture, the reaction ran to completion within an hour. Osmani et al. (2009) found that cyanidin glucosides retained 70% of their original color after one hour in a pH 7 buffer. Thus it is likely that some degradation of cyanidin and pelargonidin occurred, though this was minimized as much as possible. Another concern is the possibility of complex formation between phenolic compounds (Hidalgo et al., 2010). The possibility of these interactions or their effect on the present results cannot be discounted, as any that might have formed were not directly measured. Regardless, if such complexes did form and contributed to a synergistic or antagonistic result, this same result could be expected if the combination were consumed or used as a preservative, though possibly diminished or enhanced by the presence of other chemicals in these environments.

When using statistical analysis that correctly determines synergism or antagonism, standard errors get larger as compounds are added, making it increasingly difficult, within the error of the sampling, to show synergistic effects. This would explain why even potentially synergistic combinations (in combinations of four) were not statistically significant. In this example, only seven compounds were evaluated and focus was primarily on aglycones. Analysis could be extended, for example, to several glycoside forms of many of the compounds included (e.g. pelargonidin and cyanidin glycosides), other catechin derivatives, (epicatechin, etc.), other cinnamic acid derivatives and flavonols, and ellagitannins. Bravo (1998) concluded that the glycoside forms had significantly less antioxidant activity. By using mostly aglycones, structural elements of the core phenolic structures could be examined. This made it possible to develop a model to explain observed results using flavonoid chemistry, without blocking catechol groups used in the model. This limits extrapolation of the results to the real fruit, though it does establish a basis for the development of optimized fruit-derived antioxidant preservatives.

Conclusions for this Example

Our results show that while most of the interactions analyzed were additive, some displayed significant synergism and others demonstrated significant antagonism. A model taking into account reduction potentials, relative concentration, and the presence or absence of catechol groups explained nearly all of these results. This improves the understanding of some of the interactions that can occur in a complex environment, taking an important step toward better understanding the potential benefits of combinations, such as for food preservation.

### Example 4

### Synergistic Phytochemical Combinations Found in Strawberries

The following Table 6 shows combinations of phytochemicals found in strawberries. Also, for comparison, included are individual antioxidants and four products currently marketed for their high ORAC values. The Table 6 is ordered from highest ORAC to lowest. Values are per gram.

The most promising combination is p-coumaric acid and catechin, as they gave good results and are both readily available at low costs. Pelargonidin and quercetin-3-glucoside are more expensive, but may be available in larger quantities for significantly lower costs. Quercetin, which would be expected to have similar or better results than quercetin-3-glucoside is inexpensive.

The combinations shown here that show synergism have the potential to make a significant improvement in the quality and antioxidant power of supplements. Rather than simply combining individual fruits at random or creating concentrated extracts with unknown toxicity, the data demonstrates the power that fruit provides, while providing a very effective and safe dose.

For example: Using 1 total gram of antioxidant in a supplement would be the equivalent of about 1000 g, or 2.2 lbs, of strawberries. This would be unrealistic to consume, but a capsule containing around half this would represent an amount of fruit that could be consumed in a day, ensuring the safety of such a quantity. A capsule would also provide convenience, long-term storage and a company to stand behind their product.

TABLE 6

| | ORAC Value | |
|---|---|---|
| | (mol Trolox | Synergy (increase |
| | Equivalents/g | over sum of |
| Combination/Product Name | of mixture) | individuals) |
| cyanidin/quercetin-3-glucoside | 42,226 | 64% |
| catechin/quercetin-3-glucoside | 32,377 | 32% |
| p-coumaric acid/ | 30,420 | 50% |
| quercetin-3-glucoside | | |
| p-coumaric acid/catechin | 30,094 | 33% |
| cyanidin | 28,821 | N/A |
| catechin | 25,897 | N/A |
| cyanidin/ | 22,545 | 10% |
| quercetin-3-glucoside/ | | |
| pelargonidin | | |
| Vinomis Vindure 900 | 21,820 | N/A |
| pelargonidin | 21,710 | N/A |
| p-coumaric acid | 20,536 | N/A |
| quercetin-3-glucoside | 20,298 | N/A |
| NutraceuticsRX ORAC-15,000 ™ | 15,000 | N/A |
| High Potency Antioxidant | | |
| Nature's Answer OR AC Super 7 | 13,917 | N/A |
| p-coumaric acid/catechin/ | 11,721 | 16% |
| quercetin-3-glucoside/ | | |
| ellagic acid | | |
| p-coumaric | 11,454 | 16% |
| acid/cyanidin/quercetin-3- | | |
| glucoside/ellagic acid | | |
| cyanidin/quercetin-3- | 11,014 | 21% |
| glucoside/kaempferol/ellagic acid | | |
| p-coumaric acid/quercetin-3- | 10,839 | 17% |
| glucoside/kaempferol/ellagic acid | | |
| p-coumaric acid/ | 10,545 | 16% |
| quercetin-3-glucoside/ | | |
| ellagic acid | | |
| ellagic acid | 7,825 | N/A |
| Future Biotics Antioxidant | 4,583 | N/A |
| Superfood | | |
| cinnamon | 2,640 | N/A |
| ascorbic acid (vitamin C) | 2,000 | N/A |
| Strawberries (raw) | 35 | N/A |

TABLE 7
Current retail costs from chemical supplier Sigma:

| compound | $ | Amount | | per mg |
|---|---|---|---|---|
| Catechin | 300 | 50 | g | 0.006 |
| quercetin | 155 | 100 | g | 0.00155 |
| (aglycone) | | | | |
| quercetin-3- | 98 | 50 | mg | 1.96 |
| glucoside | | | | |
| p-coumaric acid | 68.5 | 25 | g | 0.00274 |
| kaempferol | 763 | 500 | mg | 1.526 |
| cyanidin | 54 | 1 | mg | 54 |
| pelargonidin | 131 | 10 | mg | 13.1 |
| ellagic acid | 362 | 25 | g | 0.01448 |

### Example 5

### Synergistic Potential of Fruit Ratios of Antioxidants Found in Blueberries (Vaccinium cyanococcus)

Blueberries are a rich source of antioxidants, which are thought to prevent cancer and protect the heart. Whole fruits provide a complex variety of antioxidants which likely interact, but these interactions have not been well studied, especially in whole fruit.

The antioxidant capacity of individual blueberry phenolic compounds and combinations of these compounds using oxygen radical absorbance capacity (ORAC) assays were found.

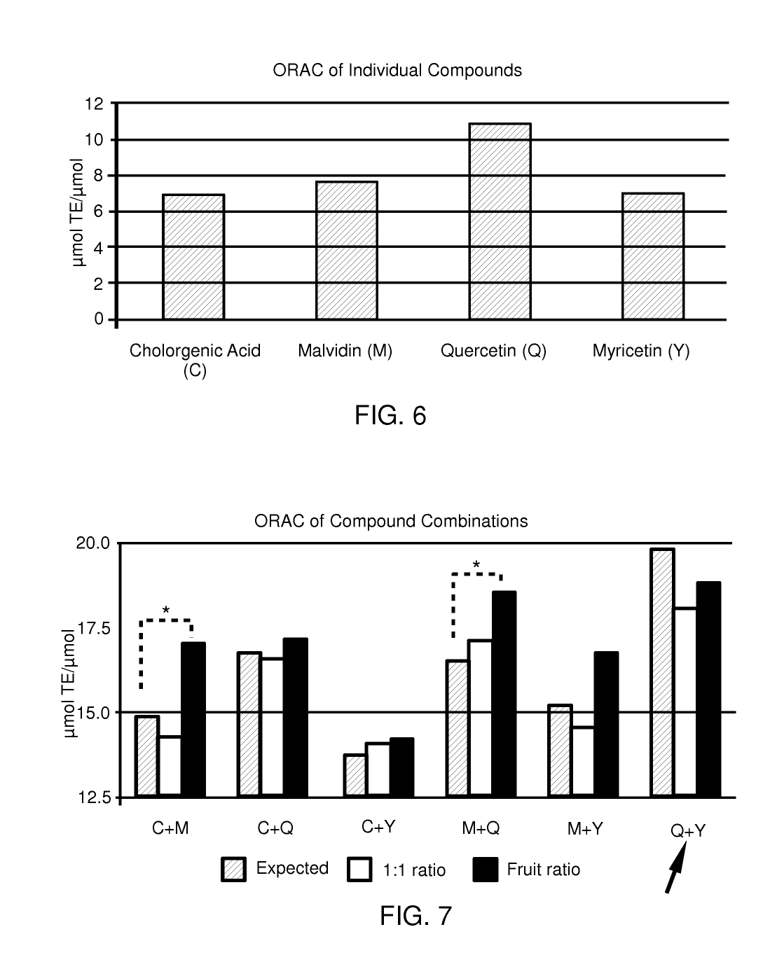

The procedures were similar to those described in Examples 1 and 3 for oranges and strawberries, respectively. Four phenolic compounds found in blueberries were selected: cholorogenic acid (C), quercetin (Q), myricetin (Y), and malvidin (M), and an ORAC assay was made of the four individual compounds (See FIG. 6). An ORAC assay was made of combinations of the four compounds at approximately 1:1 ratio, and the fruit ratios. In FIG. 7 is shown the results along with the expected value based upon additive effects of each of the compounds. A higher value indicates a synergistic effect and a lower value indicates an antagonistic effect.

The ORAC assay measures the protection of flourescein from degradation by an antioxidant or antioxidant mixture. Statistical analysis estimates the mean and standard error of the combination minus the antioxidant capacities of the individual compounds. (See FIG. 6).

Referring to FIG. 7, potential synergism was found between combinations of chlorogenic acid and malvidin, and between myricetin and quercetin. Further analysis also included combinations of three and four of malvidin, catechin, cholorogenic acid, quercetin, and myricetin, but are not shown here. However, significant synergism was found between many of these naturally occurring blueberry antioxidants. This synergism was not found when the compounds were combined at 1:1 ratios.

From this data, it can be shown that the ratio at which phenolic compounds are combined is important to whether or not that combination displays synergy or antagonism. In addition, plants have likely developed synergistic ratios in order to more effectively combat free radical damage from metabolism and UV exposure.

### Example 6

### Synergistic Phytochemical Combinations Found in Blueberries

The ORAC values for combinations of compounds in blueberries were made, using essentially the same procedure as in Example 1.

In Table 8 is shown the strongest combinations of phytochemicals found in blueberries. Values represent the ratio found in fruit unless otherwise indicated. The table is ordered from highest percent synergy to lowest. Values are per mmol of phenolic compound.

The most significant combination is catechin/chlorogenic acid/malvidin/myricetin, though malvidin is currently very expensive. The most synergistic combination not containing malvidin is chlorogenic acid/myricetin in a 1:1 ratio. The most significant combination not containing malvidin at the natural blueberry ratio is catechin/chlorogenic acid/quercetin.

The combinations that our research has demonstrated show synergism have the potential to make a significant improvement in the quality and antioxidant power of supplements. Rather than simply combining individual fruits at random or creating concentrated extracts with unknown toxicity, our data demonstrates the power that fruit and fruit antioxidants provide.

TABLE 8

| | ORAC Value (□mol | Synergy (% |
|---|---|---|
| | Trolox | increase over |
| | Equivalents/mmol of | sum of individual |
| Combination | mixture) | compounds) |
| catechin/chlorogenic acid/ | 7935 | 58% |
| malvidin/myricetin | | |
| catechin/chlorogenic acid/ | 7653 | 53% |
| malvidin | | |
| catechin/chlorogenic acid/ | 7836 | 52% |
| malvidin/quercetin | | |
| catechin/malvidin/ | 7845 | 42% |
| quercetin | | |
| catechin/malvidin | 7697 | 41% |
| catechin/malvidin 1:1 | 8278 | 39% |
| catechin/malvidin/ | 7827 | 40% |
| quercetin/myricetin | | |
| catechin/malvidin/ | 7553 | 39% |
| myricetin | | |
| malvidin/quercetin 1:1 | 7285 | 35% |
| chlorogenic acid/myricetin | 5459 | 28% |
| 1:1 | | |
| chlorogenic acid/quercetin | 6034 | 24% |
| 1:1 | | |
| malvidin/myricetin 1:1 | 5827 | 22% |
| catechin/chlorogenic acid/ | 7222 | 21% |
| quercetin | | |
| catechin/chlorogenic acid/ | 7157 | 21% |
| quercetin/myricetin | | |
| catechin/chlorogenic acid/ | 6971 | 21% |
| myricetin | | |
| catechin/myricetin 1:1 | 7910 | 21% |
| catechin/chlorogenic acid | 6767 | 16% |
| catechin/chlorogenic acid | 6294 | 16% |
| 1:1 | | |
| chlorogenic acid/ | 9122 | 15% |
| malvidin/quercetin | | |
| malvidin/quercetin | 9281 | 12% |
| malvidin/quercetin/ | 9127 | 12% |
| myricetin | | |
| malvidin/myricetin | 8386 | 10% |
| chlorogenic acid/malvidin/ | 8572 | 9% |
| quercetin/myricetin | | |
| chlorogenic acid/malvidin/ | 7979 | 8% |
| myricetin | | |
| Ratio series: | | |
| chlorogenic acid/malvidin | 4778 | 16% |
| 1:9 | | |
| chlorogenic acid/malvidin | 8524 | 14% |
| 5:13 (blueberry ratio, exp. 1) | | |
| chlorogenic acid/malvidin | 4630 | 17% |
| 5:13 (blueberry ratio, exp. 2) | | |
| chlorogenic acid/malvidin | 4958 | 34% |
| 1:1 | | |
| chlorogenic acid/malvidin | 4578 | 32% |
| 13:5 | | |
| chlorogenic acid/malvidin | 4235 | 29% |
| 9:1 | | |

What is claimed is:

1. A method of determining a composition of a nutritional-supplement with synergistic antioxidant capacity comprising:
(a) identifying antioxidant compounds in a food-stuff;
(b) measuring food-stuff ratios of at least two of the antioxidant compounds identified in the food-stuff, the food-stuff ratios being the ratios between each of the at least two compounds to each other;
(c) measuring the antioxidant capacity of the at least two antioxidant compounds;
(d) forming a mixture of the at least two antioxidant compounds at their foodstuff ratios;
(e) measuring the antioxidant capacity of the mixture;
(f) determining if the mixture has synergistic antioxidant properties by comparing the antioxidant capacity of the mixture with expected antioxidant capacity based upon the sum of the separate antioxidant capacity values of the antioxidant compounds in the mixture, synergism being shown when the antioxidant capacity is larger than the expected antioxidant capacity.

2. The method of claim 1 additionally comprising;
repeating (b), (c), (d), (e), and (f) for at least two antioxidant identified compounds where at least one of the at least two antioxidant compounds is different.

3. The method of claim 1 wherein four or more antioxidant compounds are identified and the mixture comprises a combination of at least three of the antioxidant compounds.

4. The method of claim 2 wherein at least three antioxidant compounds are identified, and the repeating is conducted for additional mixtures of possible combinations of two or three antioxidant compounds.

5. The method of claim 4 wherein the repeating is conducted for all possible mixtures of two or three antioxidant compounds.

6. A nutritional supplement comprising antioxidant compounds, the antioxidant compounds consisting essentially of two or three antioxidant compounds at ratios to each other that provide synergistic antioxidant properties.

7. The nutritional supplement of claim 6 wherein the two or three antioxidant compounds are in a ratios to each other as determined by;
(a) identifying antioxidant compounds in a food-stuff;
(b) measuring food-stuff ratios of at least two of the antioxidant compounds identified in the food-stuff, the food-stuff ratios being the ratios between each of the at least two compounds to each other;
(c) measuring the antioxidant capacity of the at least two antioxidant compounds;
(d) forming a mixture of the at least two antioxidant compounds at their foodstuff ratios;
(e) measuring the antioxidant capacity of the mixture;
(f) determining if the mixture has synergistic antioxidant properties by comparing the antioxidant capacity of the mixture with expected antioxidant capacity based upon the sum of the separate antioxidant capacity values of the antioxidant compounds in the mixture, synergism being shown when the antioxidant capacity is larger than the expected antioxidant capacity.

8. The method of claim 1 wherein the foodstuff is a fruit.

9. The method of claim 1 wherein the antioxidant capacity is measured by
oxygen radical absorbance capacity assay (ORAC),
Peroxynitrite ORAC assay (NORAC),
Hydroxyl ORAC assay (HORAC),
Oxygen Radical Absorbance Capacity pyrogallol red assay (ORAC-PG),
2,2-diphenyl-1-picrylhydrazyl radical assay (DPPH),
Ferric Reducing Ability of Plasma assay (FRAP),
Trolox Equivalent Antioxidant Capacity assay (TEAC),
Vitamin C Equivalent Antioxidant Capacity assay (VCEAC),
2′-azinobis-(3-ethylbenzothiazoline-6-sulfonic acid) assay (ABTS),
Cupric Reducing Antioxidant Capacity assay (CUPRAC),
Total Radical Trapping Antioxidant Parameter assay (TRAP), or
Cellular Antioxidant Activity assay (CAA).

10. The method of claim 1 wherein the antioxidant capacity is measured by ORAC.
